(19) United States
(12) Patent Application Publication
(10) Pub. No.: US 2024/0053358 A1
(43) Pub. Date: Feb. 15, 2024

(54) METHOD FOR ANTIBODY IDENTIFICATION FROM PROTEIN MIXTURES

(71) Applicant: ABTERRA BIOSCIENCES, INC., San Diego, CA (US)

(72) Inventors: Natalie CASTELLANA, San Diego, CA (US); Stefano BONISSONE, San Diego, CA (US); Anand PATEL, San Diego, CA (US)

(21) Appl. No.: 18/481,945

(22) Filed: Oct. 5, 2023

Related U.S. Application Data
(63) Continuation of application No. PCT/US2022/023627, filed on Apr. 6, 2022.
(60) Provisional application No. 63/201,056, filed on Apr. 9, 2021.

(51) Int. Cl. G01N 33/68; G16B 40/10; G16B 30/20; C07K 1/107

(52) U.S. Cl. CPC G01N 33/6848; G16B 40/10; G16B 30/20; C07K 1/1075

(57) ABSTRACT

Embodiments of the present disclosure relate to protein identification methods, including identification of amino acid sequences in a heterogeneous mixture of immunoglobulin and immunoglobulin-like protein molecules for reconstruction of variable regions and/or CDR3 region segments of one or more immunoglobulins.

### CROSS-REFERENCE TO RELATED APPLICATIONS

This application is a continuation application of PCT International Application Number PCT/US2022/023627, filed Apr. 6, 2022, designating the United States of America and published in the English language, which is an International Application of and claims the benefit of priority to U.S. Provisional Application No. 63/201,056, filed Apr. 9, 2021, the disclosures of which are hereby expressly incorporated by reference in their entireties.

### STATEMENT AS TO FEDERALLY SPONSORED RESEARCH

This invention was made with the support of the United States government under award number 1 R44 GM140607 by the National Institute of Health.

### REFERENCE TO SEQUENCE LISTING

The present application is being filed along with a sequence listing in electronic format. The sequence listing is provided as a file entitled ABTBI.002C1, created Oct. 5, 2023, which is 9.3 KB in size. The information in the electronic format of the sequence listing is incorporated herein by reference in its entirety.

### BACKGROUND

The present disclosure relates to protein identification methods, specifically, to identify the amino acid sequence of a heterogeneous mixture of immunoglobulin and immunoglobulin-like protein molecules to reconstruct the variable region and/or CDR3 region segments of one or more immunoglobulins.

### DESCRIPTION OF THE RELATED ART

The most directly similar existing methods for recapitulating the sequence from a monoclonal antibody sample using only proteomics exist but are limited to a purified, single, antibody or protein (U.S. Pat. No. 8,457,900). That prior work further utilizes peptide pairs rather than peptides, and uses their corresponding spectral pairs for assembly.

Similar work extends the work from U.S. Pat. No. 8,457,900 to a limited mixture, where the input is purified to be monoclonal (Guthals et al., 2015).

Other related works identify antibody variable regions/CDR3s from proteomics, but use DNA/RNA sequencing information to search against, performing database search (U.S. Patent Publ. No. 2016/0034639; U.S. Pat. No. 9,920,110). The reliance on a nucleotide (DNA or RNA derived) database is a limitation of those approaches that the current application does not possess.

De novo peptide identification is required, as no database of a priori known sequences exist. However, identified peptides require assembly into correct larger regions, e.g., antibody variable region or CDR3 region (U.S. Patent Publ. No. 2006/0020393).

Other specialty applications of polyclonal antibody analysis do not capture the sequence of the antibody, but merely classify it as a light chain for disease characterization (U.S. Patent Publ. No. 2018/0267057).

Another form of elucidating the sequence of a protein, including an antibody variable region, is using Edman degradation. However, this method will not work if the sample contains more than one protein. Further, this method cannot capture the entire antibody, and is highly unlikely to capture the CDR3 region if starting from the N-terminus as it is approximately 100 residues away.

### SUMMARY

Protein sequencing by mass spectrometry promises to uncover the true sequence of an unknown protein. Such a method is the only recourse for recovering an unknown protein without any ability to sequence nucleotides or RNA. Proteomics is typically driven by searching a mass spectrum against a database of known translated sequences, e.g., a proteome. However, many applications do not have a reliable or accurate database that will adequately represent them, such is the case with antibodies. The underlying gene segments of antibodies are encoded in the genome; however, they undergo significant somatic recombination and mutation processes, causing their significant sequence divergence from their genomically encoded origin. Furthermore, database search of mass spectra is highly sensitive to errors or variants, with only a single amino acid change preventing the correct identification with the provided template sequence. Instead, a template free approach, such as de novo, is required to identify peptides from these highly divergent and diverse proteins.

While the general nature of de novo protein sequencing could be widely applicable, the primary application has been to sequence unknown monoclonal antibodies (mAb). Such a use case arises when a hybridoma has been lost, or has diverged from its original, functional, sequence prior to nucleotide sequencing. These cases are quite common, particularly considering the sheer volume of mouse hybridomas in use.

As proteomic mAb sequencing was identified as a useful application, most studies focus on this type of protein, while some additionally tackle other types of proteins as well.

While sequencing monoclonal antibodies has proven useful, often cases arise for sequencing one or more antibodies from a polyclonal repertoire, such as multiple antibodies mixed together. Polyclonal repertoires can be obtained from serum, potentially purified against a target antigen. Often, the serum repertoire does not match well the cellular encoded repertoire, as immunoproteogenomic studies have observed. In some disease states, not only does a poor overlap exist, but the B cells encoding the serum repertoire do not reside in an accessible compartment, e.g., bone marrow. Furthermore, in most cases, timing B cell collection can be difficult, and the desired B cells can be rare, with most antibodies in the serum polyclonal going unidentified.

While the serum polyclonal repertoire contains antibodies of interest, to date, a cellular B-cell repertoire has been necessary to search mass spectra from proteomic sampling of the serum repertoire. Monoclonal antibody reconstruction methods break down when applied to a mixture of more than two or three monoclonals. The methods presented herein relate to targeted assembly of the specificity determining regions of an antibody, the CDR3 regions, from polyclonal antibody samples using only proteomic mass spectrometry data. Assessing CDR3 composition of a polyclonal repertoire can be used for identifying pathogenic autoimmune clones, or in drug discovery by recapitulating the serum clones in recombinant format.

In one embodiment, one or more complete CDR3 sequences are recapitulated from mass spectra originating from the original polyclonal sample.

In one embodiment, one or more variable region sequences are recapitulated from mass spectra originating from the original polyclonal sample.

In one embodiment, one or more full length immunoglobulin sequences are recapitulated from mass spectra originating from the original polyclonal sample.

Some embodiments provided herein relate to methods for identifying one or more immunoglobulin variable region and/or CDR3 sequences from a protein sample. In some embodiments, the methods include providing a sample containing one or more distinct peptides; obtaining mass spectra for peptides derived from the sample; identifying sequence of peptides using mass spectra alone; and assembling peptides into a larger region. In some embodiments, the assembling includes using targeted assembly of a substring. In some embodiments, the substring includes a CDR3, a V region, a full-length protein, or a substring of a full-length protein. Some embodiments provided herein relate to methods for generating peptides amenable to mass spectrometry from one or more proteins. In some embodiments, the methods include providing a sample with one or more distinct peptides; and generating peptides from the sample. In some embodiments, the peptides are generated by enzymatic digestion. In some embodiments, the enzymatic digestion includes trypsin, chymotrypsin, elastase, pepsin, Lys-C, Asp-N, Glu-C, proalanase, or thermolysin. In some embodiments, the methods further include generating peptides by way of chemical digestion. In some embodiments, the chemical digestion includes acid hydrolysis. In some embodiments, the methods further include denaturing the sample and separating antibody heavy chains from antibody light chains. In some embodiments, the antibody heavy chains and antibody light chains are separated by gel electrophoresis. In some embodiments, the distinct peptides are obtained from bands separated in a denaturing gel. In some embodiments, antibodies are denatured and digested without separation of antibody heavy chains and antibody light chains.

Some embodiments provided herein relate to methods for identifying one or more peptides from a collection of mass spectra. In some embodiments, the methods include filtering out one or more mass spectra based on features of signal and/or noise; converting each mass spectrum from the collection of mass spectra to a prefix-residue mass spectrum by a trained model; generating peptide sequence candidates; and reranking the candidates based on one or more trained models or rules. In some embodiments, the features of signal and/or noise include statistical features or information theoretic features. In some embodiments, the converting each mass spectrum from the collection of mass spectra includes filtering and removing one or more prefix-residue mass peaks; or filtering and removing one or more prefix-residue mass spectra from the collection of mass spectra. In some embodiments, the generating peptide sequence candidates includes generating a graph representation of each converted mass spectrum. In some embodiments, the generating peptide sequence candidates includes employing one or more operators to add connections between nodes and/or to increase connectivity; employing one or more operators to remove connections between nodes and/or to decrease connectivity; removing one or more nodes by filtering on model scores; adding one or more nodes based on inferred masses from di-residue or tri-residues; or optimizing scoring criteria. In some embodiments, optimizing includes a mean per node score function, geometric mean, or normalized mass score. In some embodiments, the reranking includes rescoring one or more peptides per spectrum.

Some embodiments provided herein relate to methods for assembling peptides into one or more full length proteins. In some embodiments, the methods include recruiting de novo peptides from a collection of all de novo to source and sink k-mers. In some embodiments, a target region of peptides is defined by seed source and sink k-mers; building a de Bruijn graph on k-mers of a subset of peptides; and traversing one or more paths in a graph from source to sink nodes. In some embodiments, the methods include recruiting a user-defined number of peptides wherein one seed k-mer, either source or sink is provided; performing graph construction, traversal, and validation. In some embodiments, a non-specified seed, either source or sink, are specified as all terminal nodes. In some embodiments, the methods include adding a global source node connecting to all nodes with in-degree=0. In some embodiments, both source and sink are not provided. In some embodiments, a global sink node connecting to all nodes with out-degree=0. In some embodiments, the methods further include pruning the de Bruijn graph. In some embodiments, the methods further include remapping de novo peptides to assembled sequences from either a subset or a full set of peptides. In some embodiments, the remapping reranks and filters sequenced contigs.

In some embodiments of any of the methods provided herein, the peptides are antibody proteins. In some embodiments, the antibody proteins are extracted from a sample from a subject. In some embodiments, the sample includes whole blood, serum, plasma, or other tissue. In some embodiments, the subject is a human, mouse, rat, rabbit, llama, sheep, goat, cow, shark, or other animal. In some embodiments, the subject has an adaptive immune response.

In some embodiments of any of the methods provided herein, computer generated sequences are synthesized as genetic sequences, and expressed in in vitro or cell culture expression systems.

Some embodiments provided herein relate methods for assembling peptides into one or more full length proteins. In some embodiments, the methods include initializing a first evolutionary algorithm with an initial population of peptide sequences. In some embodiments, the peptide sequences are selected from approximate, homologous, germline, or random template sequences. In some embodiments, the methods include modifying one or more candidate sequences by mutation using random variation operators. In some embodiments, one parent sequence produces one offspring sequence. In some embodiments, the methods include evaluating one or more candidate sequences with a fitness function by mapping a source selected from peptide evidence, k-mer evidence, substrings of peptides, or any combination thereof. In some embodiments, the methods further include initializing a second evolutionary algorithm for assembling a different region of the one or more candidate proteins. In some embodiments, the initial population includes a result of a de Bruijn graph assembly. In some embodiments, the initial population includes an overlap graph assembly result. In some embodiments, the overlap graph assembly result is produced from peptides identified by any of the methods described herein. In some embodiments, the initial population includes a result of germline sequences. In some embodiments, the initial population includes randomly generated sequences. In some embodiments, the initial population includes an initial population of CDR3s that includes a result of germline sequences with random sequences. In some embodiments, the methods further include generating expected-length CDR3 sequences from the result of germline sequences with random sequences. In some embodiments, the initial population includes a result of CDR3 sequences generated from peptides recruited by tags and random sequences. In some embodiments, the methods further include generating expected length CDR3 sequences from the peptides recruited by tags and random sequences. In some embodiments, the one or more candidate sequences include one protein sequence that include one or more regions. In some embodiments, the one or more candidate sequences include two or more protein sequences that include one or more regions each. In some embodiments, the evolutionary algorithm employs elitism. In some embodiments, the evolutionary algorithm does not employ elitism. In some embodiments, the methods further include applying random variation operators to modify one or more candidate sequences by crossover. In some embodiments, two parents produce one or two offspring sequences.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 illustrates a flowchart of an exemplary process, starting from mass spectra originating from polyclonal antibody sample, to sequencing of variable regions and/or CDR3 regions, in accordance with an embodiment of the present disclosure. These resulting regions can be merged and further refined to obtain one or more refined variable/CDR3 regions, and finally one or more of the sequences can be synthesized, expressed, and tested for function similar to that defined in the input polyclonal sample.

FIG. 2 illustrates a flowchart for an exemplary method of targeted CDR3 assembly, in accordance with an embodiment of the present disclosure. Recruitment of likely CDR3 covering PSMs (right), which is used as input to the CDR3 assembly algorithm, detailed (left).

FIG. 3 illustrates a pipeline for de novo peptide sequencing, as performed by Riptide, or other algorithms, methods, and software, in accordance with an embodiment of the present disclosure.

FIG. 4 illustrates an example de Bruijn graph for sequences PEPTIDE and PEPSIDE with different types of scoring metadata used for traversal, pruning, and assembly, in accordance with an embodiment of the present disclosure.

FIGS. 5A-5B illustrate an example method for peptide mapping for trastuzumab using database search (FIG. 5A) and a de Bruijn graph (FIG. 5B) with larger value of k=7, showing the loss in sensitivity and disconnected nature of the graph, hindering correct traversal, in accordance with an embodiment of the present disclosure.

FIG. 6 illustrates targeted assembly of the heavy chain CDR3 for trastuzumab case study, in accordance with an embodiment of the present disclosure. The CDR3 graph with four, highly similar, possible paths is shown.

FIGS. 7A-7B illustrate targeted assembly of heavy chain CDR3 in mixture of rabbit monoclonals, showing the CDR3 graph with 30 possible paths (FIG. 7A); the top 4 scoring paths without any gaps in remapped peptide coverage, and no single residue/ambiguous mass variants (FIG. 7B; top); and remapped de novo peptides to contigs 15 and 3 (FIG. 7B; middle and bottom, respectively).

FIGS. 8A-8B illustrate targeted assembly of heavy chain CDR3 in Llama single-domain polyclonal purified against KLH, showing: the CDR3 graph with 30 possible paths (FIG. 8A); the top 4 scoring paths without any gaps in remapped peptide coverage, and no single residue/ambiguous mass variants (FIG. 8B).

FIG. 9 illustrates an exemplary method for evolutionary algorithm-based assembly of full length sequences or partial sequences (FIG. 9 panel a); the representation with multiple regions corresponding to framework (FWR) and complementarity determining regions (CDR) (FIG. 9 panel b); the representation focusing assembly on the CDR3 region as an example of focused assembly (FIG. 9 panel c); intra-region crossover of two parents, exemplifying single-point crossover (FIG. 9 panel d); inter-region crossover of two parents, exemplifying swapping an entire region (FIG. 9 panel e).

FIG. 10 illustrates a run of the evolutionary algorithm in targeted CDR3 search of four sequences using the weighted fitness function with α=0.25. Plotted is the fitness by generation across 30 independent runs.

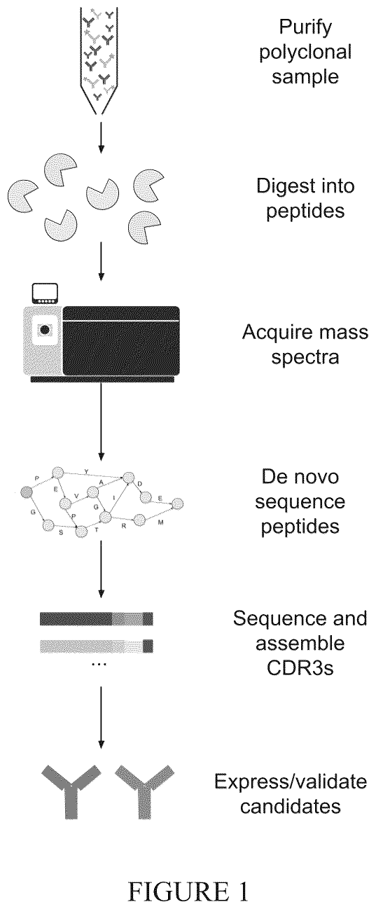

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Monoclonals/polyclonals were buffer exchanged and concentrated using 5 kDa MWCO filter (Corning Spin-X) and quantified by Qubit fluorometer (Life Technologies). The resulting fraction was selected and run in multiple lanes on a reducing SDS-PAGE. Gel bands for heavy and optionally light chains were excised and in-gel digested using trypsin (Promega), chymotrypsin (Promega), elastase (Promega), and pepsin (Worthington). Digested peptides were individually subjected to LC-MS/MS with either 1 hr (trastuzumab case study and mixture case study) or 2 hr gradients (llama polyclonal case study) on an Orbitrap Fusion Lumos Tribid (ThermoFisher). MS/MS was acquired in data-dependent precursor selection in HCD/EThcD doublet fragmentation mode.

Additionally, for the llama polyclonal case study, polyclonal was purified from serum of final bleed using KLH antigen conjugated to NHS-activated agarose resin (ThermoFisher). Columns were washed three times with 1×PBS buffer. Antibody bound to the column was eluted with 20 mL of 0.25M glycine HCl pH 1.85 elution buffer into 20 fractions. Fractions were buffer exchanged and concentrated using 5 kDa MWCO filter (Corning Spin-X) and quantified by Qubit fluorometer (Life Technologies). Affinity purified IgGs were separated into each sub-isotype by passing them through a protein G column (which binds IgG1 and IgG3). Flow-through contains IgG2, while elution at different pHs separate IgG1 and IgG3.

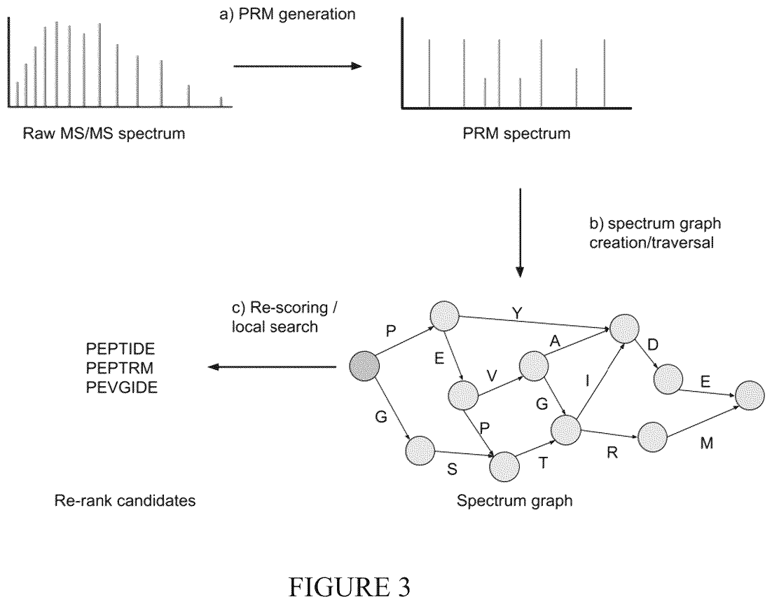

An internal de novo mass spectrum sequencer, Riptide, was used to sequence all raw mass spectra. The algorithm for Riptide has not been described previously, but briefly, it operates on a deconvoluted spectrum or a spectrum of charge 2. A random forest model is used to assign scores to prefix-residue masses (PRMs), creating a PRM spectrum from the input spectrum. This PRM spectrum is then converted into a spectrum graph, which is pruned, and traversed by heaviest path from the 0 Da node to the node of size of the parent mass. The path that is maximized is one with the highest the average PRM (represented by nodes) score. Paths and/or peptide sequences output from the traversal can then optionally be rescored and/or reranked by a different scoring method, e.g., spectral probability, percent intensity explained, percentage of fragments identified, by a specially trained re-ranking model, etc. The general process for sequencing a peptide from a mass-spectrum is outlined in FIG. 3.

The feature vector for the PRM model was composed by binning ±50 Da windows around the prefix p¹ and suffix s¹ charge 1 ions, and ±25 Da windows around p² and s² charge 2 ions. Binning for low-resolution models can be performed using 1 Da bins. The values within each bin are the rank normalized intensities of any peaks falling within that bin. Three additional spectrum level features are added as well, totaling 307 features. Hi-resolution instruments allow for matching fragments at tighter ∈-tolerances, e.g., 0.01 Da. However, applying the same binning procedure described above from the 1 Da bin resolution at a 0.01 Da resolution generates 30,003 features. This explosion of dimensionality is prohibitive for building an accurate model; feature selection (such as choosing a subset of original features) must be performed prior to learning. Feature selection was performed to reduce the dimensionality for the hi-resolution feature vector representation. Univariate ranking of features using signal-to-noise ratio was done to downselect to the top 100 binned features. The statistic is defined for a feature i over classes c={0,1}:

$$s {n}_{i} = \frac{\vert {μ}_{0} - {μ}_{1} \vert}{\vert {σ}_{0} + {σ}_{1} \vert} \qquad ( 1 )$$

Similar features were obtained when optimizing for entropy rather than signal-to-noise ratio, and similar features were also obtained when using random forest's variable selection features to downselect to 100 features.

The transformation of the raw peaks [symbol] , to PRM peaks [symbol] _{P} is a critical step. If done naively, one can consider a set of fragment ions [symbol] ={b,y,b²,y²,b−H2O, b−NH3,y−H2O,y−NH3,a,x}, where [symbol] _{P} is created by applying each fragment ion function ƒ∈ [symbol] to each peak p∈ [symbol] ; [symbol] _{P}={p′=ƒ(p)|∀p∈ [symbol] ∧∀ƒ∈ [symbol] }. This is the procedure for naive PRM generation, generating a PRM spectrum with | [symbol] _{P}|=|S|−| [symbol] | peaks.

In some embodiments, [symbol] _{P} is created in the same manner, but then applied to the random forest model over every p′∈ [symbol] _{P}, changing the intensity of p′ to the score of the model, yielding a PRM spectrum [symbol] _{P}; now, p∈ [symbol] _{\hat{P}} are 0≤p≤1. For contrast, other algorithms apply a log transformation on the intensity of peaks p′∈ [symbol] _{P} instead of a model probability. While | [symbol] _{P}|=| [symbol] _{\hat{P}}|, a threshold is applied, η, to peaks in [symbol] _{\hat{P}} removing low probability prefix peaks, thereby reducing noise, and subsequently reducing the complexity of the induced spectrum graph. [symbol] _{\hat{P}}(η)={p∈ [symbol] _{\hat{P}}|p≥η}. Alternatively, a sliding window filter can be applied to the PRM spectrum [symbol] _{P}.

Starting from a PRM spectrum [symbol] _{P}, as defined above, the PRM spectrum ideally contains only prefix masses of neutral charge. A spectrum graph is a graph G=(V,E) where V={v|v∈ [symbol] _{P}}. Special nodes at 0 and the parent mass (pm) are added to V. The edge set E={(u,v)∈V×V|(v−u)∈_{τ} Σ}, for a given alphabet of amino acids, Σ. Matching a mass difference as a residue r in the alphabet, r∈_{τ}Σ, is dependent on an error tolerance T. Several values for τ were tested, but hi-resolution instruments can use small tolerances, and r=0.01 Da is used throughout the text. Traversal of the spectrum graph from node 0 to the parent mass node pm provides a path that represents a potentially complete candidate peptide sequence for the spectrum.

The spectrum graph creation (as described above) connects two peaks if their difference equals to an amino acid mass, within an ∈-tolerance. Before creating the graph, the input peak set is pruned, and peaks that are very close to each other (within an ∈-tolerance) are merged into a single peak. This reduces the complexity of the graph. A heaviest path traversal of the graph from node 0 to node pm is performed, retaining the top k paths. Additional pruning based on topological features was tested, but was deemed as too aggressive at removing nodes. Every node (representing a peak in [symbol] _{P}) u∈V has a mass(u) and prmScore(u). As the graph is traversed in topological order, length(u) and score(u) are updated for each node u∈V. The score for a node v∈V can be defined by a function over source nodes of all incoming edges in(v). An online computation of the mean of scores along a path would be defined as:

$$score ( v ) = \frac{p r m S c o r e ( v ) - {max}_{u ∈ i n ( v )} score ( u )}{length ( v )} + {max}_{u ∈ i n ( v )} score ( u ) \qquad ( 2 )$$

Other on-line computations and means can be used, and tested were on-line geometric mean, a mass normalized aggregate score, and summation. The arithmetic mean and geometric mean had the best performances across a variety of datasets.

One common problem is that PRMs can be missed, and thus not included in the spectrum graph. This can happen for a variety of reasons: 1) PRM scoring model predicts poorly; 2) the PRM mass was not considered; 3) the scoring model does not consider charges states that contain evidence for the PRM. If the cause was 1 or 3, then a solution would be to improve the PRM predictive model. However, if the cause was the second reason, for example not considering the peak, then the model never had a chance to predict, and the graph is disconnected when it potentially could be rescued. One strategy to recover missed prefix masses is to attempt to identify missed intermediate fragments based on di-residue mass differences, the di-residue alphabet Σ₂=Σ×Σ. This requires identifying mass differences between all pairs of nodes, u, v∈V such that v−u∈Σ₂. Once such pairs of nodes (u,v) are identified to belong to a di-residue difference r∈Σ₂, then two intermediate masses w₁ and w₂ are created such that w₁=u+r₁ and w₂=u+r₂, where r₁ is one of the two residues of r, while r₂ is the other, and w₁, w₂∈Σ. Both w₁ and w₂ must be considered since the order or the di-residues in r cannot be determined only from the mass difference v−u.

The two new putative PRMs w₁ and w₂ are then classified using the PRM model, and retained if their score is sufficiently high. If either is retained, it is connected to the graph by adding edges (u,w₁), (w₁,v) for w₁, and (u,w₂), (w₂,v) for w₂. This procedure can be done for all pairs of nodes, but is particularly important for u=0 and for v=pm, as it is more common for peaks at ends of the spectrum to be missing.

Probabilities from learned models are not necessarily well-calibrated, for example a predicted model probability of 0.8 actually reflects a fraction of 80% of positives for that sample. Not all model training procedures ensure this property. Luckily, a simple approach to correction is to learn a model that takes as input the model output probability, and outputs a corrected probability. Platt scaling performs this correction by using a logistic regression model for this probability re-calibration. Well calibrated models are essential for interpreting model probabilities as true probabilities, as they can be used as a per-residue confidence score. Additionally, combining outputs from two or more models requires that the scores be distributed similarly in the output space. Calibrated probabilities ensure this, and allow for straightforward merging, something that is required for combining multiple acquisitions from different modes, e.g., HCD/ETD. Well calibrated probabilities are not described as a feature of other de novo tools, despite it being critical for the proper merging of multiple spectra across acquisition types (see Merging multiple acquisition modes). Logistic regression calibration models were trained, one for each PRM model, on a separate dataset from which the random forest PRM model was trained.

Since Riptide requires a model to predict PRM quality given a fragmented spectrum, in this case a random forest model, this model is specific to the type of spectral acquisition used, and a separate model is used for each type of acquisition, e.g., one model for HCD, another for EThcD, another for ETD, etc. Ions with multiple acquisition modes, e.g., doublet HCD/EThcD spectra, are combined after each PRM spectrum has been created, using a simple union operator and merging peaks that are within an ∈-tolerance.

Merging spectra from multiple acquisition modes (e.g., HCD and ETD) can provide complementary patterns of ion fragmentation. Few tools support this feature, while the most recent algorithms do not support such a feature. Merging of doublet or triplet spectra can potentially be performed at multiple steps: 1) prior to any processing, 2) after PRM spectrum creation, or 3) after spectrum graph creation. Merging prior to processing has the disadvantage of not being able to determine which fragment ion types should be used in PRM creation. Merging after spectrum graphs have been created would require adding edges between existing nodes between the two graphs, complicating the merging procedure. Instead, merging is performed after PRM spectrum creation but before spectrum graph creation. Merging at this step, and using PRM models specific to each acquisition mode, is enabled only due to the model probability calibration.

The de novo sequencing task interprets spectra from peptides a wide range of peptide lengths from 6 to 20 or more amino acids. Longer peptides are more difficult to sequence de novo, and reliable sequences are rarely found for lengths greater than 15. A logistic regression model is used to re-score de novo interpretations to calculate correctness probabilities that better compares peptide spectrum matches between different spectra. Briefly, two feature sets were evaluated for improved scoring: 1) de novo peptide score and parent mass, and 2) de novo peptide score and number of paths in the spectrum graph from the 0 node to parent mass node. The de novo peptide score is the average PRM score for the top ranked peptide interpretation.

The path count in the spectrum graph is efficiently computed by first calculating C(v), the number of paths to any PRM node v starting from 0 node. Since the spectrum graph is a directed acyclic graph, the C(v)=Σ_{(u,v)∈E} C(u) where node u appears before v, and E are the edges the spectrum graph. The path count is a proxy complexity for the PRM spectrum, and difficulty in finding correct de novo sequences. To find the best proxy, different parameters for spectrum graph construction were evaluated; minimum prm score filtering 0.2 and 0.5, mass error tolerance 0.01 Da, 0.003 Da, and permitting edges between two PRM nodes if the mass difference is within error tolerance of two amino acids.

Identification of peptide sequences can be performed using other de novo sequencing approaches.

Antibody specific peptides can be further helped by identification by weighting l-mers on the graph that are more frequently observed in antibody sequences.

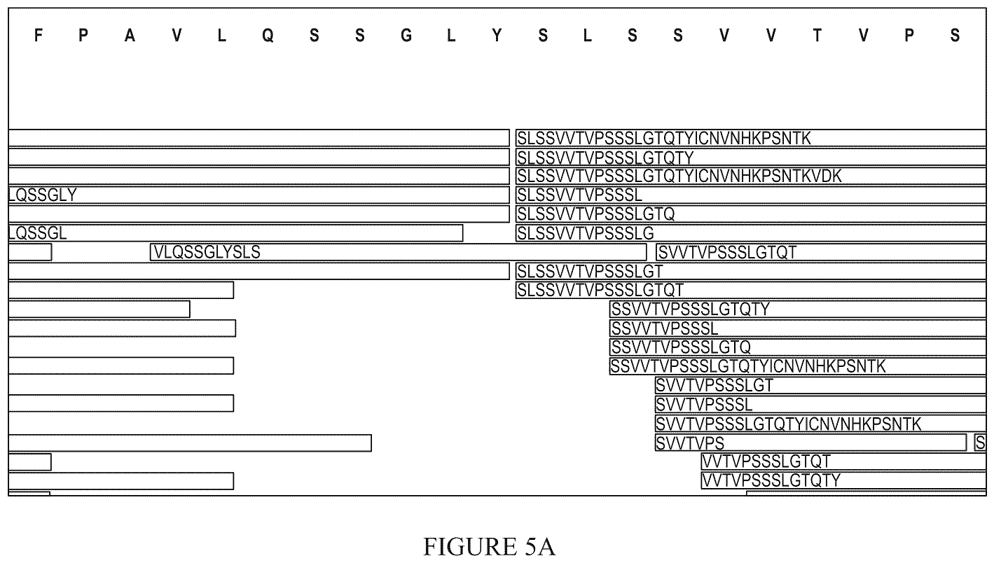

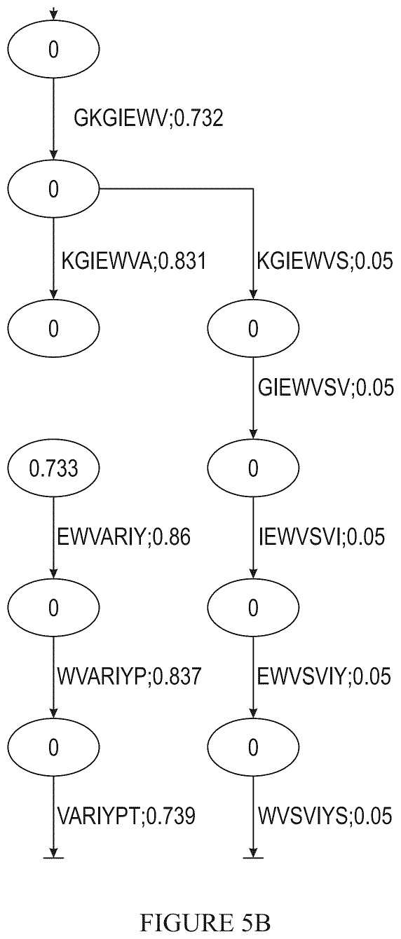

To target the CDR3 region, one of two approaches must be taken: a) select a subset of peptides/spectra that cover the CDR3 region; b) build the representative data structure (e.g., de Bruijn graph, string graph, etc.) and focus in on the CDR3 region. While approach b seems appealing, to properly build a de Bruijn graph that can span the CDR3 a small k must be used. If not using a small k, then the graph is likely to be disconnected due to non-uniformity in peptides due to enzyme specificity, e.g., FIGS. 5A and 5B. The exemplary sequence shown in FIG. 5A is FPAVLQSSGLYSLSSVVTVPS (SEQ ID NO: 20), with various sequences and portions of sequences shown therein as SEQ ID NOs: 21-39. If built on all spectra with small k as is needed for sensitivity, a large, highly connected graph will ensue, which will be difficult to prune to just the region of interest. Instead, method a was employed where a rough filter of de novo identified peptides suspected of covering the CDR3 region were selected. This filtering was done recursively from the N-term of the CDR3 downstream, as well as from the C-term of the CDR3 upstream. An N-term tag and C-term tag are used to bootstrap the recruitment of PSMs, and are later used as source/sink in the graph (detailed subsequently).

The union of these two sets of de novo peptide-spectrum matches (PSMs) were used by the de Bruijn graph to construct a CDR3 graph. A de Bruijn graph G=(V,E) is defined over k-mers, strings from Σ^{k}, obtained from de novo sequenced peptides. Each k-mer is split up into the first (k−1)-mer and the second (k−1)-mer, the set of all (k−1)-mers derived from a set of peptide strings P comprise the nodes in V. For each k-mer, the first (k−1)-mer, u∈V, and the second (k−1)-mer, v∈V, are connected by a directed edge (u,v)∈E. This builds a graph G such that any node has at most |Σ| outgoing and incoming edges to it.

Once a graph G is created, it can be traversed using a standard longest path algorithm. Additional information can be added to G to alter how the graph is traversed. For example, abundance information of each (k−1)-mer can be included at each node (or k-mer on each edge), and then the heaviest path algorithm that optimizes the coverage can be used.

$${v}_{score} = {v}_{count} + {max}_{u ∈ i n ( v )} + {u}_{score} \qquad ( 3 )$$

For abundance v_{count} and scores from previous nodes u_{score} from all nodes from incoming edges (v). The score from equation (3) maximizes the path of (k−1)-mers that have the most coverage, which may or may not be the best approach. For genomic sequencing, where coverage is expected to be constant, this is a reasonable approach. However, for peptides where spectral acquisition can vary greatly, it is not ideal.

Instead of abundance v_{count} (such as spectral count), it can be replaced with a model-derived score of the (k−1)-mer. Every peptide p∈ [symbol] that contains v, has representative quality values w^{p}, where w₁^{p} represents the score of position 1 in the (k−1)-mer, w₂^{p} position 2, etc. The average residue score of the constituent residues in a (k−1)-mer v across all peptides P, is then the average of all quality positions, across all instances of peptides containing v, p(v):

$${v}_{score} = \frac{1}{p ( v )} {\sum }_{p ∈ 𝒫} {I}_{v ⊆ p} ( \frac{1}{k - 1} {\sum }_{i = 1}^{k - 1} {w}_{i}^{p} ) + {max}_{u ∈ i n ( v )} + {u}_{score} \qquad ( 4 )$$

Where the indicator function I_{v⊂ p} is 1 if the (k−1)-mer v is contained in peptide p, 0 otherwise. Equation (4) is similar to that from Tran et al., 2016. The score of each node is the average residue score over (k−1)-mer residues, averaged over multiple peptides that contain (k−1)-mer. This averaging can mask poor scoring single residues. A third, improved, representation is to replace the average of residue scores over the (k−1)-mer with the average single residue score. This type of single residue meta-data represents the last character from a k-mer, and is represented on the edge (u,v). This ends up changing the update rule to:

$${v}_{score} = \frac{1}{p ( v )} {\sum }_{p ∈ 𝒫} {I}_{v ⊆ p} ′ {w}_{k - 1}^{p} + {max}_{u ∈ i n ( v )} + {u}_{score} \qquad ( 5 )$$

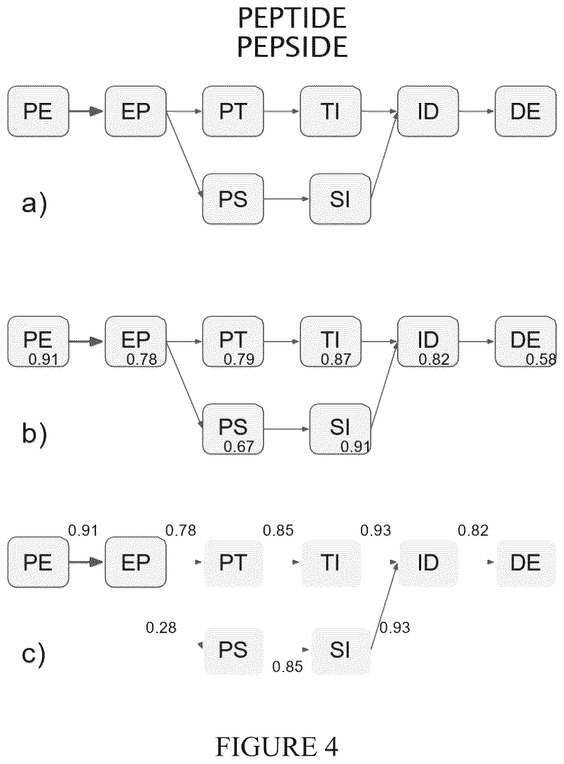

All three types of meta-data configurations are depicted in FIG. 4 for the same set of two peptides. The figure shows for the same topology, how two different paths could be selected depending on the representation of weights on the nodes or edges.

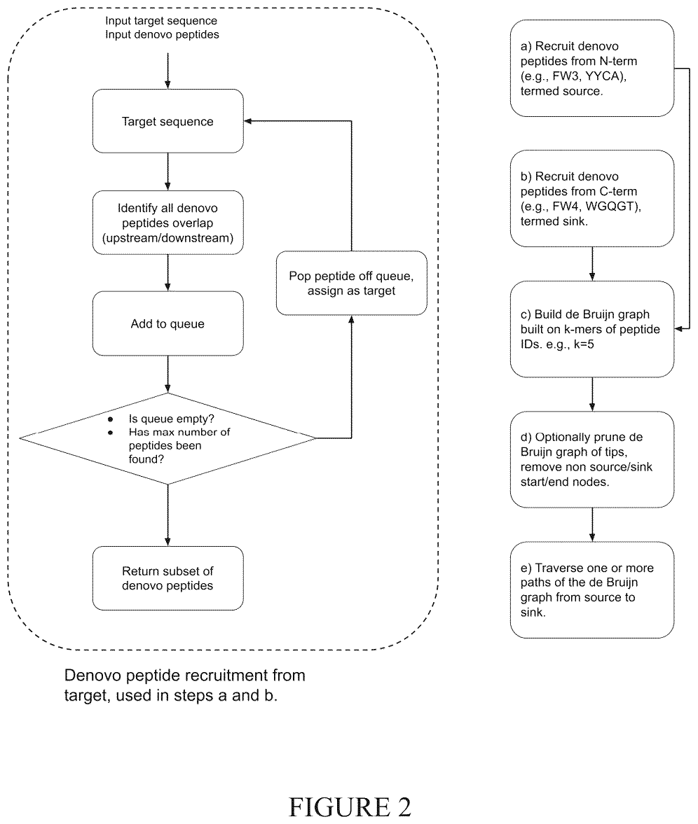

The CDR3 graph is then pruned by: tips are clipped; any cycles are broken heuristically; non source/sink terminal nodes are removed recursively until a graph with only the tag source and sink remain. The resulting graph is then traversed using a heaviest path algorithm find either all paths from source to sink, or the top m scoring paths, for a user-defined value of m. The resulting contigs then have de novo PSMs remapped to them, selecting the top scoring candidates. Either full peptides can be mapped to contigs using pairwise alignment, or exact matching l-mers. Both were tested, with l-mers being used, where l>k. Specifically, contigs with identical monoisotopic masses are reduced to only the top scoring sequence based on mean coverage of remapped PSMs. The described pipeline is shown in FIG. 2.

Contigs can also be produced using the iterative de Bruijn graph approach. For a range of kₗ to kₕ, construct a CDR3 graph with kₕ, and produce contigs as described elsewhere herein. Repeat the process with decreasing k until k=kₗ, and report unique candidate contigs. The iterative graph approach can be used to find correct contigs, as too large a k may result in correct sequences having broken paths in the graph, and too small a k may result in high scoring false chimeric paths.

As used herein, the term “k-mer” refers to substrings of a length k contained within a sequence. For example, a k-mer may refer to an amino acid sequence that is a length of “k.” For example, a 5-mer may be a substring that is 5 amino acids long.

As used herein, a “source” node refers to a node that has a suffix that overlaps with the prefix of a “sink” node. For example, in a directed edge (u,v), u is a source node (for example source read or source k-mer) and v is a sink node (for example sink read or sink k-mer).

As used herein, “prune” or “pruning” refers to removing segments of a graph. For example, thinly traversed, superfluous, and/or spurious elements such as nodes, branches, loops, edges, etc. may be removed from the graph, and the graph may be reconstructed without the removed elements.

As used herein “elitism” or “elitist” refers to an algorithm which allow the best organism(s) (“elites”) from the current generation to carry over to the next, unaltered. For example, “elite” individuals are not expelled from the active gene-pool of the population (such as the subsequent initial population) in favor of worse individuals.

The assembly process produces one or more candidate contigs. These candidates can be true sequences, and also likely, false sequences often comprised of chimeric sequences; such as part of a true sequence from one origin integrated into a true sequence from another origin. This is a common pitfall with genomic and transcriptomic assembly as well. To determine which contig(s) are correct in genomic assemblies, reads are remapped to the putative contigs, providing support from the basal data structure, the reads, for specific branching paths selected by the assembler. A similar idea is applied to protein assemblies from peptide reads; remap de novo peptides, termed re novo, back to the candidate sequence. This provides similar evidence for specific branching and variants, as done in genomic assemblies.

Any method to reconstruct one or more antibody sequences from a polyclonal sample should be able to recapitulate a full-length sequence from a purified monoclonal. To test this, a reagent version of the trastuzumab monoclonal antibody was sequenced (Absolute Antibody cat. no. Ab00103-10.0).

Accordingly, some embodiments provided herein relate to the following enumerated embodiments:
1. A method for identifying one or more immunoglobulin variable region and/or CDR3 sequences from a protein sample, the method comprising: providing a sample containing one or more distinct antibody proteins; obtaining mass spectra for peptides derived from the sample; identifying sequences of peptides from the mass spectra; and assembling peptides into a region.
2. The method of embodiment 1, wherein the assembling comprises using targeted assembly of a substring.
3. The method of embodiment 2, wherein the substring comprises a CDR3, a V region, a full-length protein, or a substring of a full-length protein.
4. A method for generating peptides amenable to mass-spectrometry from one or more proteins, the method comprising: providing a sample with one or more distinct peptides; and generating peptides from the sample.
5. The method of embodiment 4, wherein the peptides are generated by enzymatic digestion.
6. The method of embodiment 5, wherein the enzymatic digestion comprises trypsin, chymotrypsin, elastase, pepsin, Lys-C, Asp-N, Glu-C, ProAlanase, or thermolysin.
7. The method of any one of embodiments 5-6, further comprising generating peptides by chemical digestion.
8. The method of embodiment 7, wherein the chemical digestion comprises acid hydrolysis.
9. The method of any one of embodiments 4-8, further comprising denaturing the sample to form antibody heavy chains and antibody light chains.
10. The method of embodiment 9, further comprising separating the antibody heavy chains from the antibody light chains.
11. The method of embodiment 10, wherein the antibody heavy chains and the antibody light chains are separated by gel electrophoresis.
12. The method of any one of embodiments 4-9, wherein the one or more distinct peptides are obtained from bands separated in a denaturing gel.
13. The method of any one of embodiments 4-7, wherein the distinct peptides are obtained from denaturation and digestions of antibodies in solution.
14. A method for identifying one or more peptides from a collection of mass spectra, the method comprising: filtering one or more mass spectra from the collection of mass spectra based on features of signal and/or noise; converting each mass spectrum from the collection of mass spectra to a prefix-residue mass spectrum by a trained model; generating peptide sequence candidates; and reranking the candidates based on one or more trained models or rules.
15. The method of embodiment 14, wherein the features of signal and/or noise comprise statistical features or information theoretic features.
16. The method of any one of embodiments 14-15, wherein converting each mass spectrum from the collection of mass spectra comprises: filtering and removing one or more prefix-residue mass peaks; or filtering and removing one or more prefix-residue mass spectra from the collection of mass spectra.
17. The method of any one of embodiments 14-16, wherein generating peptide sequence candidates comprises: generating a graph representation of each converted mass spectrum.
18. The method of any one of embodiments 14-17, wherein generating peptide sequence candidates comprises: employing one or more operators to add connections between nodes and/or to increase connectivity; employing one or more operators to remove connections between notes and/or to decrease connectivity; removing one or more nodes by filtering on model scores; adding one or more nodes based on inferred masses from di-residue or tri-residues; or optimizing scoring criteria, wherein optimizing comprises a mean per node score function, geometric mean, or normalized mass score.
19. The method of any one of embodiments 14-18, wherein reranking comprises rescoring one or more peptides per spectrum.
20. A method for assembling peptides into one or more full length proteins, the method comprising: recruiting de novo peptides from a collection of all de novo to source and sink k-mers, wherein a target region of peptides is defined by seed source and sink k-mers; building a de Bruijn graph on k-mers of a subset of peptides; and traversing one or more paths in a graph from source to sink nodes; or recruiting a user-defined number of peptides wherein one seed k-mer, either source or sink, is provided; performing graph construction, traversal, and validation, wherein a non-specified seed, either source or sink, were specified as all terminal nodes; or adding a global source node connecting to all nodes with in-degree=0, wherein both source and sink are not provided, and wherein a global sink node connecting to all nodes with out-degree=0.
21. The method of embodiment 20, further comprising pruning the de Bruijn graph.
22. The method of any one of embodiments 20-21, further comprising remapping de novo peptides to assembled sequences from either a subset or a full set of peptides, wherein the remapping reranks and filters sequenced contigs.
23. The method of embodiment 22, wherein the assembled sequences are antibody proteins.
24. The method of embodiment 23, wherein the antibody proteins are extracted from a sample from a subject.
25. The method of embodiment 24, wherein the sample comprises whole blood, serum, plasma, cerebrospinal fluid, or other tissue.
26. The method of any one of embodiments 24-25, wherein the subject is a human, mouse, rat, rabbit, llama, alpaca, camel, sheep, goat, cow, horse, chicken, shark, or other animal.
27. The method of any one of embodiments 24-26, wherein the subject has an adaptive immune response.
28. The method of any one of embodiments 1-27, further comprising synthesizing computer-generated sequences as genetic sequences.
29. The method of any one of embodiments 1-28, further comprising expressing genetic sequences in in vitro or cell culture expression systems.
30. A method for assembling peptides into one or more full length proteins, the method comprising: initializing a first evolutionary algorithm with an initial population of peptide sequences selected from approximate, homologous, germline, or random template sequences; modifying one or more candidate sequences by mutation using random variation operators, wherein one parent sequence produces one offspring sequence; and evaluating one or more candidate sequences with a fitness function by mapping a source selected from peptide evidence, k-mer evidence, substrings of peptides, or any combination thereof.
31. The method of embodiment 30, further comprising initializing a second evolutionary algorithm for assembling a different region of the one or more candidate proteins.
32. The method of any one of embodiments 30-31, wherein the initial population comprises a result of a de Bruijn graph assembly.
33. The method of any one of embodiments 30-32, wherein the initial population comprises an overlap graph assembly result.
34. The method of embodiment 33, wherein the overlap graph assembly result is produced from peptides identified by the method of any one of embodiments 14-18.
35. The method of any one of embodiments 30-34, wherein the initial population comprises a result of germline sequences.
36. The method of any one of embodiments 30-35, wherein the initial population comprises randomly generated sequences.
37. The method of any one of embodiments 30-36, wherein the initial population comprises an initial population of CDR3s comprising a result of germline sequences with random sequences.
38. The method of embodiment 37, further comprising generating expected-length CDR3 sequences from the result of germline sequences with random sequences.
39. The method of any one of embodiments 30-38, wherein the initial population comprises a result of CDR3 sequences generated from peptides recruited by tags and random sequences.
40. The method of embodiment 39, further comprising generating expected length CDR3 sequences from the peptides recruited by tags and random sequences.
41. The method of any one of embodiments 30-40, wherein the one or more candidate sequences comprise one protein sequence comprising one or more regions.
42. The method of any one of embodiments 30-41, wherein the one or more candidate sequences comprise two or more protein sequences comprising one or more regions each.
43. The method of any one of embodiments 30-42, wherein the evolutionary algorithm employs elitism.
44. The method of any one of embodiments 30-42, wherein the evolutionary algorithm does not employ elitism.
45. The method of any one of embodiments 30-44, further comprising applying random variation operators to modify one or more candidate sequences by crossover, wherein two parents produce one or two offspring sequences.

### EXAMPLES

Some aspects of the embodiments discussed above are disclosed in further detail in the following examples, which are not in any way intended to limit the scope of the present disclosure.

### Example 1: Targeted Assembly of Peptides

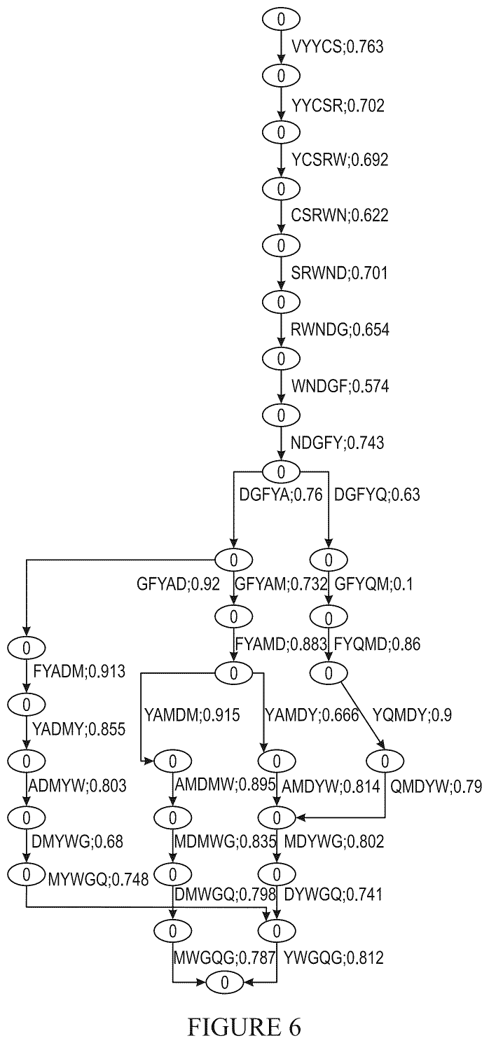

Targeted assembly of the CDR3 proceeded by searching for source and sink tags of DTAVYYC (SEQ ID NO: 1) and WGQGTIV (SEQ ID NO: 2), respectively. A compact graph with only four highly similar paths was constructed. It is expected that a monoclonal would generate a relatively simple graph, as variants should only occur based on de novo peptide sequencing errors rather than true variation. FIG. 6 shows the resulting de Bruijn graph and three assembled contigs with remapped coverage. The top scoring sequence by remapped mean positional PSM coverage corresponds to the true sequence of trastuzumab (with one GG→N ambiguity). The two remaining sequences are minor variants, as seen by a reduction in coverage at those variant positions, due to de novo peptide sequencing errors.

This shows the robustness of the approach to peptide sequencing errors, that the true sequence is able to be obtained even when incorrect PSMs are incorporated into the de Bruijn graph. Furthermore, the re novo approach to filtering true contigs from false ones proved successful.

The simplest simulation of a polyclonal sample is a mixture of monoclonal antibodies. Six monoclonal antibodies were mixed in equimolar concentration, one of the six is known a priori as it is the rabbit anti-beta galactosidase antibody described previously. This mixture was subjected to the same enzyme digests and mass spectrometry analysis to generate spectra; subsequently, de novo peptides were obtained by Riptide.

Assembling the CDR3s for the rabbit antibody mixture was done by using the following source tag TYFCA (SEQ ID NO: 3) and sink tag GTIVTV (SEQ ID NO: 4), as the N-term tag is conserved across all V genes of rabbits, while a variant in framework 4 exists in WG[QP]GTIV (SEQ ID NO: 5), so the conserved downstream tag GTIVTV (SEQ ID NO: 4) is used.

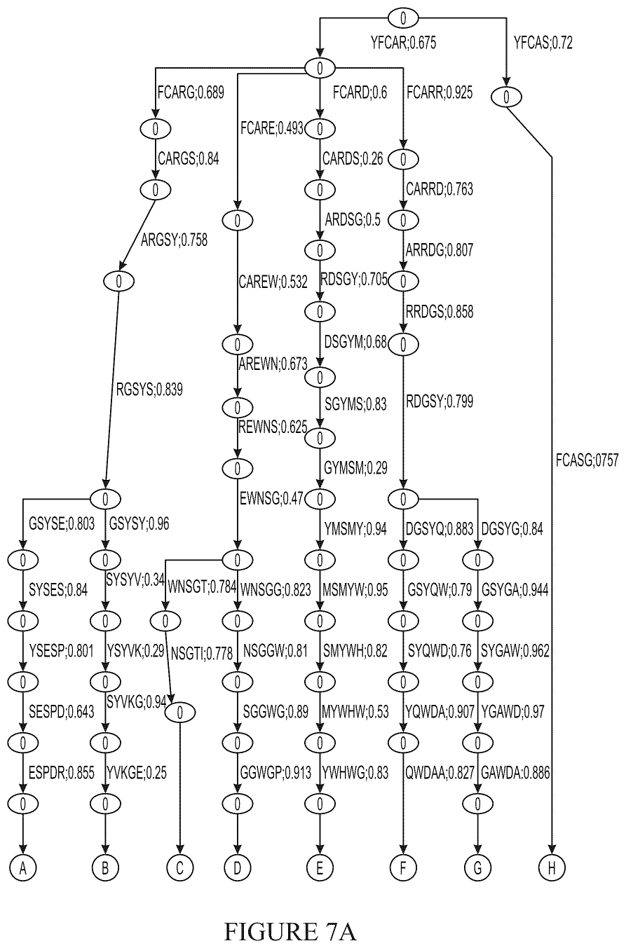

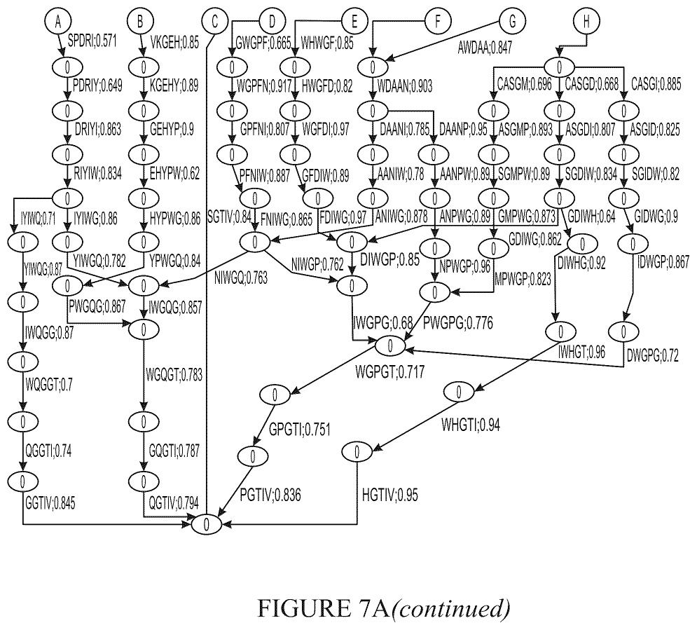

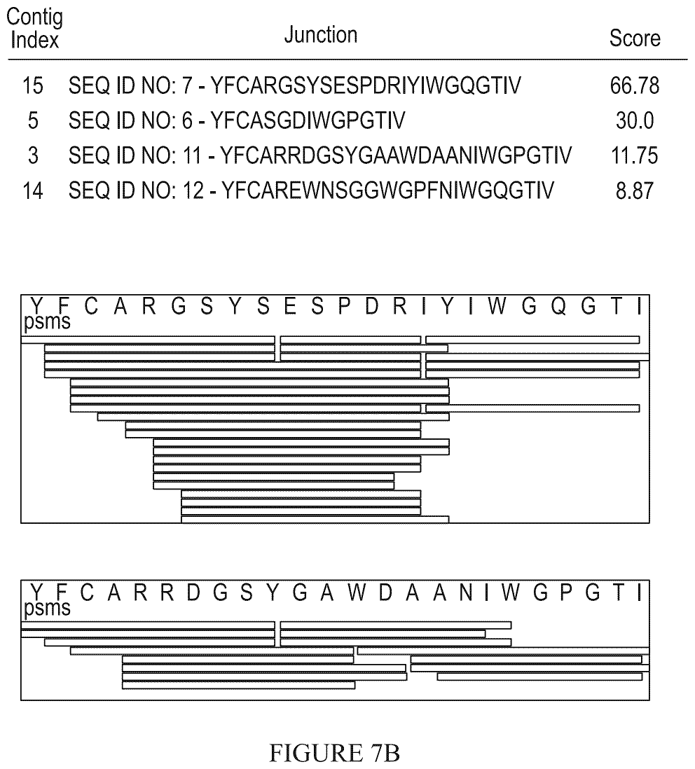

The graph produced 12 different non-ambiguous mass sequences, some with variants in the framework 4 variant noted previously. The pruned graph is shown in FIG. 7A, with the top 4 shown in FIG. 7B. The remainder of the 12 candidates not shown were poorer scoring variants of those shown.

With the exception of the shortest CDR3 sequence YFCASGDIWGPGTIV (SEQ ID NO: 6), no single spectrum contains the entire CDR3 region, requiring assembly across multiple peptides. This is often a limitation of trypsin digestion, with R/K residues often at one or more locations in the CDR3. Chymotrypsin produces smaller peptides due to commonly observed Y/F/W residues, and other enzymes have similar problems. Combining enzymes has allowed assembly of monoclonal antibodies.

The one known antibody CDR3 from, YFCARGSYSESPDRIYIWGQGTIV (SEQ ID NO: 7), was perfectly assembled and recapitulated from this mixture.

Polyclonal sera from an immunized llama was enriched for single domain antibodies (sdAbs), either IgG2 or IgG3 isotype. This enriched polyclonal was further purified against the KLH antigen, resulting in an antigen specific sdAb polyclonal. The purified serum was then subjected to enzyme digests with four distinct enzymes and subjected to mass spectrometry analysis. Peptides were identified from the mass spectra using Riptide, and the resulting PSMs were analyzed to assemble the CDR3 as described in elsewhere herein methods of Targeted CDR3 assembly.

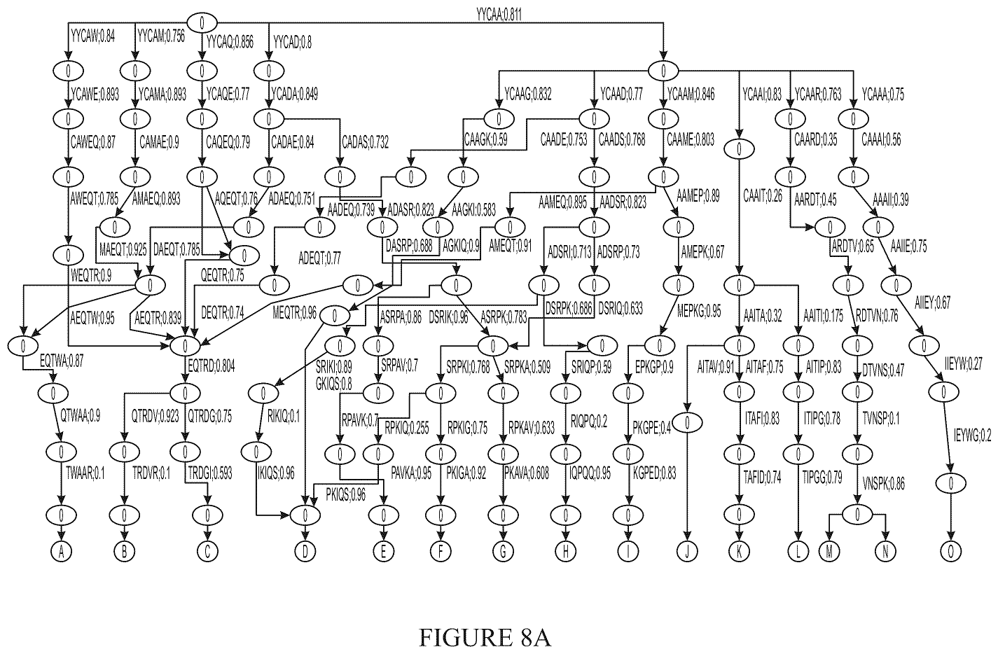

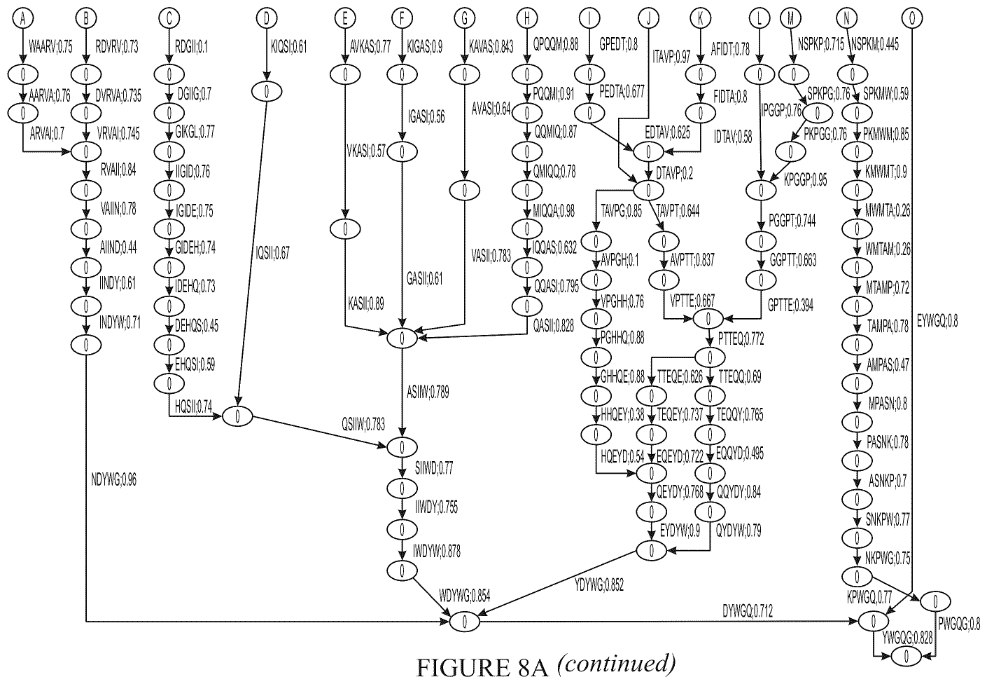

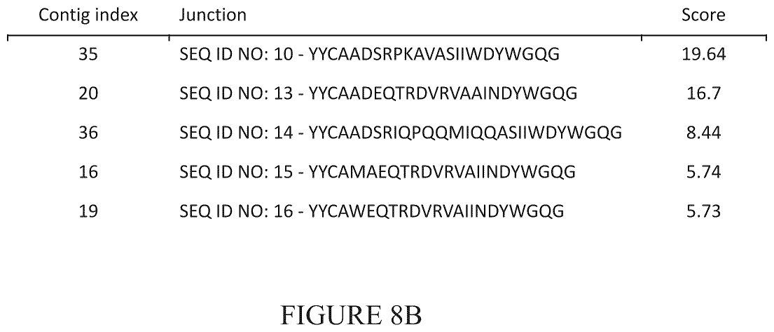

The following parameters were used to build the CDR3 de Bruijn graph: source tag VYYCA (SEQ ID NO: 8), sink tag WGQGT (SEQ ID NO: 9), k=5. Peptides from PSMs were remapped back to find contigs using l-mers with l=7. FIG. 8A shows the pruned de Bruijn graph of the CDR3s, and FIG. 8B shows the top contigs with remapped peptide coverage assigned per position. Traversal of the graph and subsequent filtering for contigs with identical mass resulted in 30 distinct contigs (by sequence).

The top scoring remapped contig, YYCAADSRPKAVASIIWDYWGQG (SEQ ID NO: 10), was found exactly in the related cellular NGS repertoire. This remapping of de novo contigs to NGS provides a high level of confidence in the hit, since the NGS was not used for searching the mass spectra (not limiting the space of identifiable sequences), and that it sampled from an orthogonal biological source, e.g., B cells rather than serum antibodies.

An alternative method for antibody assembly using meta-heuristic search is described, specifically utilizing an evolutionary algorithm (EA). In this formulation, given de novo peptide sequences, and one or more template sequences, the EA progresses by: a) creating an initial population; b) applying random variation operators; c) computing fitness of each individual; d) applying selection of parents and offspring to create the next generation of the population; e) repeating steps b-d for N generations. This process is a general EA structure (e.g., U.S. Pat. No. 5,255,345). The parts specific to antibody assembly from peptides are in the i) representation of the individual; ii) random variation operators; iii) fitness function computation; iv) initial population method. Each one is described in subsequent paragraphs.

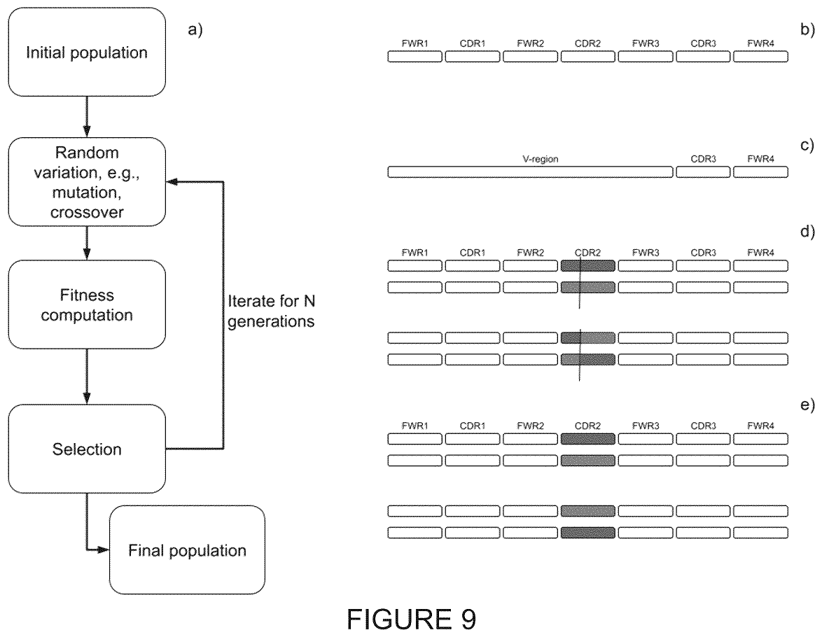

Candidate representation is important for how the optimization progresses. An antibody chain sequence is divided into contiguous regions, the simplest being framework regions (FWR) and complementarity determining regions (CDR), resulting in 7 regions per chain sequence. To search for N different sequences simultaneously, there are then N*7 regions, with each 7 considered a distinct antibody. To search only for CDR3 regions, we can simplify it to 3 regions per antibody: FWR1-FWR3, CDR3, FWR4. These two types of representations are shown in FIG. 9 panel b and panel c. Other delineations of regions are feasible as well, and the EA described can operate on any definition of region.

Random variation is problem specific, and for antibody assembly two forms may be used: mutation and crossover. Mutation generates one offspring from one parent, while crossover results in either one or two offspring from two parents. Mutation operators can substitute, insert, or delete one residue at a time. The mutation rate can be region specific, e.g., CDR regions can have a higher mutation rate (resulting in more of the search space traversed) than FWR regions, enabling more efficient search. Crossover operators can be subdivided into three types: 1) intra-region; 2) inter-region; 3) inter-sequence. Intra-region can perform single point, double point, or uniform crossover within the same region of two candidates. Inter-region can swap two entire regions from different parent individuals. Inter-sequence can swap two entire sequences between two individuals, this is only useful if searching for N>1 sequences.

Fitness evaluation of candidate individuals quantifies, as a numerical value, how well the candidate solution represents the provided input peptide sequences; the better the fit of peptides to the candidate sequences, the better the solution, e.g., if maximizing fitness the larger the fitness the better the solution. An appropriate fitness function must be used for the EA to converge on the correct solution. A fitness function uses de novo peptides mapped to the candidate sequence, and computes a numerical value to summarize the coverage of peptides on the candidate sequence. There are two approaches to mapping de novo peptide evidence to a candidate sequence: a) k-mer based mapping; b) peptide based mapping. Mapping k-mers allows approximate mapping of peptides by considering fixed sized k-mers and performing exact mapping of k-mers to a candidate sequence. Mapping k-mers allows for de novo peptides with errors to produce valid k-mers to map to the candidate. Full de novo peptide mapping to the candidate sequence is not tolerant to errors, but provides more direct evidence of spectral information and counts. Mapping k-mers is faster than full peptide mapping, which translates to faster EA simulations, or use of EA on larger sequence populations, simulations with more generations, or more simulation repetitions.

For k-mer mapping, given a vector of k-mer coverage x^{k} where xᵢ^{k}=number of k-mers mapping to position i of n positions, indicator function Iₐ₌₀={1 if a=0; 0 otherwise, and penalties Δ_{Sp} and Δ₃ₚ, then the fitness can be defined as:

$$f ( x ) = \frac{1}{n} {\sum }_{i}^{n} {x}_{i}^{k} - \frac{1}{n} {\sum }_{i}^{n} {I}_{{x}_{i}^{k} = 0} + {Δ}_{5 p}^{k} + {Δ}_{3 p}^{k}$$

Similarly, if peptides are exactly mapped, a vector of exact mapping coverage x^{e} where xᵢ^{e}=number of peptides exactly mapping to position i, and have a similar function to evaluate fitness. A weighted combination can also be defined with weight α:

$$f ( x ) = ( \frac{1}{n} {\sum }_{i}^{n} {x}_{i}^{k} ) α + ( \frac{1}{n} {\sum }_{i}^{n} {x}_{i}^{e} ) ( 1 - α )$$

$$- ( \frac{1}{n} {\sum }_{i}^{n} {l}_{{x}_{i}^{k} = 0} ) α + ( \frac{1}{n} {\sum }_{i}^{n} {I}_{{x}_{i}^{e} = 0} ) ( 1 - α )$$

$$+ {Δ}_{5 p}^{k} ′ α + {Δ}_{5 p}^{e} ( 1 - α )$$

$$+ {Δ}_{3 p}^{e} α + {Δ}_{5 p}^{e} ( 1 - α )$$

In the above definitions, Iₐ₌₀ is an indicator function as described above, 5p/3p penalties Δ_{Sp} and Δ₃ₚ, respectively, and 0≤α≤1.

The initial population can be created in a variety of methods. The simplest is random initialization, however, this typically performs poorly due to being placed in a plateau in the fitness landscape. Other methods that can help bootstrap the EA and were tested include starting from an inferred germline template. For monoclonals this is easily done by mapping de novo peptides to each germline combination of V, D, J, and C genes, note D genes can be omitted and replaced with a placeholder sequence, e.g., YYYYYYYYYYY (SEQ ID NO: 17). The combination of germlines that has the most coverage, mapping peptides, or best fitness function score can be selected. Random variants of this germline can then be created by mutating residues to form the initial population. This is extended to multiple antibodies by identifying the top M germlines and replicating them to fill the desired number of proteins to search. Yet other methods to initialize the starting population is to utilize assembled contigs from de Bruijn graph or overlap graph based assemblies. All these methods described are also used for initializing the population when searching for subregions, e.g., CDR3 search.

Another method is to identify ends of the region in question, e.g., if CDR3 is targeted, identifying peptides that contain FW3 region matches and their downstream sequence can be combined with peptides matching the FW4 region tag and their upstream region. For example, if AYYC is the end FW3, then peptides matching AYYCXXXX (SEQ ID NO: 18) can be identified by alignment, and the XXXX sequence can be used as a partial start. Similarly, FW4 tags can be searched, e.g., WGQGT (SEQ ID NO: 9), so that peptides with matches and upstream sequence, e.g., ZZZZWGQGT (SEQ ID NO: 19), can include the relevant upstream sequence ZZZZ for use as partial ends. Then, the set of all partial starts and ends can be used to create randomly combined sequences and filling-in ambiguous characters with randomly chosen amino acids up to desired lengths, to produce an initial population of sequences.

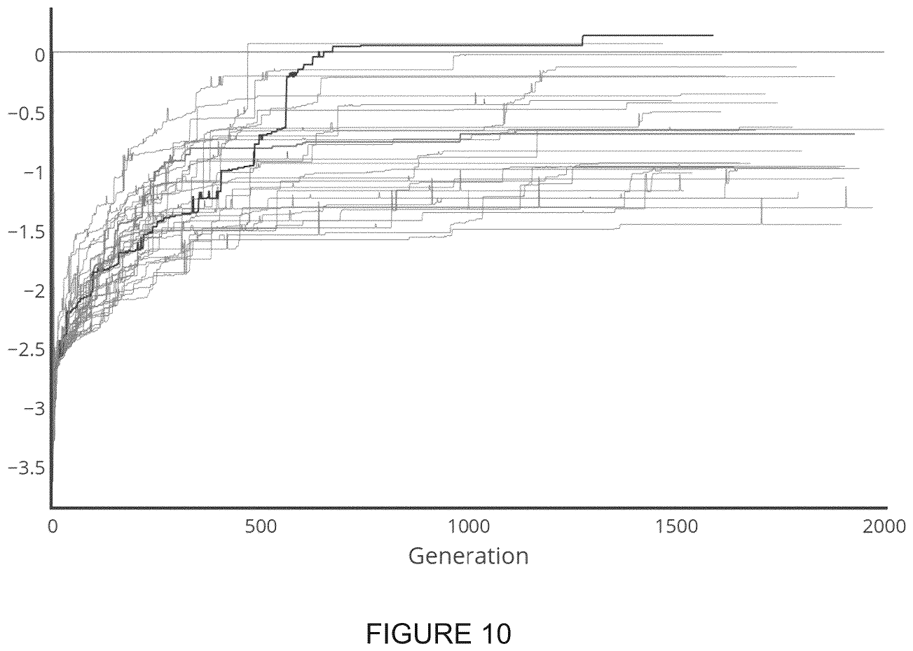

CDR3s from the rabbit antibody mixture were assembled by using the evolutionary algorithm described above with the following design choices. A representation focusing on CDR3 was used, depicted in FIG. 9 panel c, with four sequences in each individual. The V region and FW4 were selected as the best germline according to coverage of mapping de novo identified peptides. The end of the V region was TYFC and the beginning of the FW4 region was WGQG. The CDR3 region of the initial population was initialized with random sequences derived from de novo peptides generated as described elsewhere herein. Mutation, intra-region crossover, and inter-region crossover were employed, with values of 0.0357 and 0.25 for mutation and both crossover operators, respectively. The fitness function defined herein using both forms of evidence was used, with α=0.25. The EA was run independently thirty times, each time reporting the best individual using elitism. Plots of convergence are shown in FIG. 10. The best individual of the runs contained three of four CDR3s as reported with the de Bruijn graph, including the one known sequence YFCARGSYSESPDRIYIWGQGTIV (SEQ ID NO: 7).

All publications, patents, and patent applications mentioned in this specification are herein incorporated by reference to the same extent as if each individual publication, patent, or patent application was specifically and individually indicated to be incorporated by reference.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

What is claimed is:

1. A method for identifying one or more immunoglobulin variable region and/or CDR3 sequences from a protein sample, the method comprising:
providing a sample containing one or more distinct antibody proteins;
obtaining mass spectra for peptides derived from the sample;
identifying sequences of peptides from the mass spectra; and
assembling peptides into a region.

2. The method of claim 1, wherein the assembling comprises using targeted assembly of a substring.

3. The method of claim 2, wherein the substring comprises a CDR3, a V region, a full-length protein, or a substring of a full-length protein.

4. A method for generating peptides amenable to mass-spectrometry from one or more proteins, the method comprising:
providing a sample with one or more distinct peptides; and
generating peptides from the sample.

5. The method of claim 4, wherein the peptides are generated by enzymatic digestion.

6. The method of claim 5, wherein the enzymatic digestion comprises trypsin, chymotrypsin, elastase, pepsin, Lys-C, Asp-N, Glu-C, ProAlanase, or thermolysin.

7. The method of claim 5, further comprising generating peptides by chemical digestion.

8. The method of claim 7, wherein the chemical digestion comprises acid hydrolysis.

9. A method for identifying one or more peptides from a collection of mass spectra, the method comprising:
filtering one or more mass spectra from the collection of mass spectra based on features of signal and/or noise;
converting each mass spectrum from the collection of mass spectra to a prefix-residue mass spectrum by a trained model;
generating peptide sequence candidates; and
reranking the candidates based on one or more trained models or rules.

10. The method of claim 9, wherein the features of signal and/or noise comprise statistical features or information theoretic features.

11. The method of claim 9, wherein converting each mass spectrum from the collection of mass spectra comprises:
filtering and removing one or more prefix-residue mass peaks; or
filtering and removing one or more prefix-residue mass spectra from the collection of mass spectra.

12. The method of claim 9, wherein generating peptide sequence candidates comprises:
generating a graph representation of each converted mass spectrum.

13. A method for assembling peptides into one or more full length proteins, the method comprising:
recruiting de novo peptides from a collection of all de novo to source and sink k-mers, wherein a target region of peptides is defined by seed source and sink k-mers;
building a de Bruijn graph on k-mers of a subset of peptides; and
traversing one or more paths in a graph from source to sink nodes; or
recruiting a user-defined number of peptides wherein one seed k-mer, either source or sink, is provided;
performing graph construction, traversal, and validation, wherein a non-specified seed, either source or sink, were specified as all terminal nodes; or
adding a global source node connecting to all nodes with in-degree=0, wherein both source and sink are not provided, and wherein a global sink node connecting to all nodes with out-degree=0.

14. The method of claim 13, further comprising pruning the de Bruijn graph.

15. The method of claim 13, further comprising remapping de novo peptides to assembled sequences from either a subset or a full set of peptides, wherein the remapping reranks and filters sequenced contigs.

16. The method of claim 15, wherein the assembled sequences are antibody proteins.

17. A method for assembling peptides into one or more full length proteins, the method comprising:
initializing a first evolutionary algorithm with an initial population of peptide sequences selected from approximate, homologous, germline, or random template sequences;
modifying one or more candidate sequences by mutation using random variation operators, wherein one parent sequence produces one offspring sequence; and
evaluating one or more candidate sequences with a fitness function by mapping a source selected from peptide evidence, k-mer evidence, substrings of peptides, or any combination thereof.

18. The method of claim 17, further comprising initializing a second evolutionary algorithm for assembling a different region of the one or more candidate proteins.

19. The method of claim 17, wherein the initial population comprises a result of a de Bruijn graph assembly.

20. The method of claim 17, wherein the initial population comprises an overlap graph assembly result.


SEQUENCE LISTING (39 sequences)

SEQ ID NO: 1 - protein, 7 aa, synthetic construct
  REGION 1..7: note = Synthetic
DTAVYYC

SEQ ID NO: 2 - protein, 7 aa, synthetic construct
  REGION 1..7: note = Synthetic
WGQGTIV

SEQ ID NO: 3 - protein, 5 aa, synthetic construct
  REGION 1..5: note = Synthetic
TYFCA

SEQ ID NO: 4 - protein, 6 aa, synthetic construct
  REGION 1..6: note = Synthetic
GTIVTV

SEQ ID NO: 5 - protein, 8 aa, synthetic construct
  REGION 1..8: note = Synthetic
WGQPGTIV

SEQ ID NO: 6 - protein, 15 aa, synthetic construct
  REGION 1..15: note = Synthetic
YFCASGDIWGPGTIV

SEQ ID NO: 7 - protein, 24 aa, synthetic construct
  REGION 1..24: note = Synthetic
YFCARGSYSESPDRIYIWGQGTIV

SEQ ID NO: 8 - protein, 5 aa, synthetic construct
  REGION 1..5: note = Synthetic
VYYCA

SEQ ID NO: 9 - protein, 5 aa, synthetic construct
  REGION 1..5: note = Synthetic
WGQGT

SEQ ID NO: 10 - protein, 23 aa, synthetic construct
  REGION 1..23: note = Synthetic
YYCAADSRPKAVASIIWDYWGQG

SEQ ID NO: 11 - protein, 26 aa, synthetic construct
  REGION 1..26: note = Synthetic
YFCARRDGSYGAAWDAANIWGPGTIV

SEQ ID NO: 12 - protein, 24 aa, synthetic construct
  REGION 1..24: note = Synthetic
YFCAREWNSGGWGPFNIWGQGTIV

SEQ ID NO: 13 - protein, 24 aa, synthetic construct
  REGION 1..24: note = Synthetic
YYCAADEQTRDVRVAAINDYWGQG

SEQ ID NO: 14 - protein, 28 aa, synthetic construct
  REGION 1..28: note = Synthetic
YYCAADSRIQPQQMIQQASIIWDYWGQG

SEQ ID NO: 15 - protein, 24 aa, synthetic construct
  REGION 1..24: note = Synthetic
YYCAMAEQTRDVRVAIINDYWGQG

SEQ ID NO: 16 - protein, 23 aa, synthetic construct
  REGION 1..23: note = Synthetic
YYCAWEQTRDVRVAIINDYWGQG

SEQ ID NO: 17 - protein, 11 aa, synthetic construct
  REGION 1..11: note = Synthetic
YYYYYYYYYYY

SEQ ID NO: 18 - protein, 8 aa, synthetic construct
  REGION 1..8: note = Synthetic
  VARIANT 5..8: note = Xaa = Any Amino Acid
AYYCXXXX

SEQ ID NO: 19 - protein, 9 aa, synthetic construct
  REGION 1..9: note = Synthetic
  VARIANT 1..4: note = Xaa = Any Amino Acid
XXXXWGQGT

SEQ ID NO: 20 - protein, 21 aa, synthetic construct
  REGION 1..21: note = Synthetic
FPAVLQSSGLYSLSSVVTVPS

SEQ ID NO: 21 - protein, 30 aa, synthetic construct
  REGION 1..30: note = Synthetic
SLSSVVTVPSSSLGTQTYICNVNHKPSNTK

SEQ ID NO: 22 - protein, 18 aa, synthetic construct
  REGION 1..18: note = Synthetic
SLSSVVTVPSSSLGTQTY

SEQ ID NO: 23 - protein, 33 aa, synthetic construct
  REGION 1..33: note = Synthetic
SLSSVVTVPSSSLGTQTYICNVNHKPSNTKVDK

SEQ ID NO: 24 - protein, 13 aa, synthetic construct
  REGION 1..13: note = Synthetic
SLSSVVTVPSSSL

SEQ ID NO: 25 - protein, 16 aa, synthetic construct
  REGION 1..16: note = Synthetic
SLSSVVTVPSSSLGTQ

SEQ ID NO: 26 - protein, 14 aa, synthetic construct
  REGION 1..14: note = Synthetic
SLSSVVTVPSSSLG

SEQ ID NO: 27 - protein, 14 aa, synthetic construct
  REGION 1..14: note = Synthetic
SVVTVPSSSLGTQT

SEQ ID NO: 28 - protein, 15 aa, synthetic construct
  REGION 1..15: note = Synthetic
SLSSVVTVPSSSLGT

SEQ ID NO: 29 - protein, 17 aa, synthetic construct
  REGION 1..17: note = Synthetic
SLSSVVTVPSSSLGTQT

SEQ ID NO: 30 - protein, 16 aa, synthetic construct
  REGION 1..16: note = Synthetic
SSVVTVPSSSLGTQTY

SEQ ID NO: 31 - protein, 11 aa, synthetic construct
  REGION 1..11: note = Synthetic
SSVVTVPSSSL

SEQ ID NO: 32 - protein, 14 aa, synthetic construct
  REGION 1..14: note = Synthetic
SSVVTVPSSSLGTQ

SEQ ID NO: 33 - protein, 28 aa, synthetic construct
  REGION 1..28: note = Synthetic
SSVVTVPSSSLGTQTYICNVNHKPSNTK

SEQ ID NO: 34 - protein, 12 aa, synthetic construct
  REGION 1..12: note = Synthetic
SVVTVPSSSLGT

SEQ ID NO: 35 - protein, 10 aa, synthetic construct
  REGION 1..10: note = Synthetic
SVVTVPSSSL

SEQ ID NO: 36 - protein, 27 aa, synthetic construct
  REGION 1..27: note = Synthetic
SVVTVPSSSLGTQTYICNVNHKPSNTK

SEQ ID NO: 37 - protein, 7 aa, synthetic construct
  REGION 1..7: note = Synthetic
SVVTVPS

SEQ ID NO: 38 - protein, 13 aa, synthetic construct
  REGION 1..13: note = Synthetic
VVTVPSSSLGTQT

SEQ ID NO: 39 - protein, 14 aa, synthetic construct
  REGION 1..14: note = Synthetic
VVTVPSSSLGTQTY